(19) United States
(12) Patent Application Publication
(10) Pub. No.: US 2012/0065683 A1
(43) Pub. Date: Mar. 15, 2012

(54) INTERSPINOUS PROCESS DISTRACTION DEVICE

(76) Inventors: Fan-Ching Kuo, Taipei City (TW); Shu-Hui Yang, Taipei City (TW); Yi-Chen Kuo, Taipei City (TW)

(21) Appl. No.: 12/880,848

(22) Filed: Sep. 13, 2010

(51) Int. Cl. A61B 17/70

(52) U.S. Cl. 606/248

(57) ABSTRACT

An interspinous process distraction device includes a curved plate which is formed with an upper plate and an opposite lower plate by bending a board material, with the upper plate being penetrated with two upper wire holes and the lower plate being penetrated with two lower wire holes; an upper fixing wire, two ends of which penetrate from bottom to top the two upper wire holes of the upper plate to be tied on a spinous process; and a lower fixing wire, two ends of which penetrate from top to bottom the two lower wire holes of the lower plate to be tied on the spinous process. This interspinous process distraction device is provided with a low manufacturing cost and high durability.

### BACKGROUND OF THE INVENTION

a) Field of the Invention

The present invention relates to an interspinous process distraction device, and more particularly to an interspinous process distraction device which can stably support a spine and is provided with a low manufacturing cost as well as high durability.

b) Description of the Prior Art

A spine is an essential part of a human skeleton, serving as a backbone of the skeleton to support muscles and organs in an upper half of the human body and linking with a lower half.

The spine is formed by many vertebrae and an interior of each vertebra is passed through by many critical nerves. As a person gets older, the spine is aged or an accidental injury occurs, the vertebra can be injured that spacing between two adjacent vertebrae is shortened to compress the nerves, which harms the nerves and induces numbness as well as a feel of pain.

An existing conventional structure includes an adjustable interspinous process distraction device which contains a curved plate formed with an upper plate and an opposite lower plate by bending a board material. The upper plate is penetrated with a screw hole and a stud is assembled in the screw hole. A bottom end of the stud is abutted at the lower plate and by tightening the stud at the lower plate, a distance between the upper plate and the lower plate can be changed, thereby providing a stable support to the vertebrae.

However, for the aforementioned distraction device which uses the stud and the screw hole, the structures are too complicated and an upper and lower vertebra of a patient can be easily lack of an elastic movement after using the stud, which results in a too high cost of the distraction device. In addition, there is also an issue of inferior applicability.

### SUMMARY OF THE INVENTION

Accordingly, the primary object of the present invention is to provide an interspinous process distraction device which can stably support a spine, and is provided with a low cost as well as high durability.

To achieve the aforementioned object, the interspinous process distraction device of the present invention, includes a curved plate which is formed with an upper plate and an opposite lower plate, with the upper plate being penetrated with two upper wire holes and the lower plate being penetrated with two lower wire holes; an upper fixing wire, two ends of which penetrate respectively from bottom to top the two upper wire holes of the upper plate to be tied on a spinous process; and a lower fixing wire, two ends of which penetrate respectively from top to bottom the two lower wire holes of the lower plate to be tied on the spinous process.

For the aforementioned adjustable interspinous process distraction device, end parts and opposite exterior sides of the upper and lower plates are separated and protruded respectively with two upper clamp pieces and a lower clamp piece to be clamped on the spinous processes.

For the aforementioned adjustable interspinous process distraction device, the exterior sides of the upper and lower plates are formed with inverted hooks.

Two ends of the aforementioned upper fixing wire are sheathed with a wire sleeve and two ends of the lower fixing wire are sheathed with a wire sleeve.

The adjustable interspinous process distraction device of the present invention is at least provided with following advantages and functional improvements:
1. Compared to the prior art where many vertebrae are tied and positioned together, the present invention only positions two adjacent spinous processes at a time; therefore, a movement between the vertebrae, excessive stiffness of the spine or a pain which can be easily formed at the spine by a slight movement can be avoided, thereby providing better comfortableness.
2. Compared to the existing interspinous process distraction device, the present invention eliminates the structures of screw hole and stud; therefore, the high cost due to that the structures are too complicated can be avoided. In addition, tooth collapse to the screw hole under many times of adjustment can be prevented.
3. The present invention can provide the stable support to two adjacent vertebrae, thereby achieving an object of intensifying the skeleton structure.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a three-dimensional schematic view of the present invention.

FIG. 2 shows a three-dimensional exploded view of the present invention.

FIG. 3 shows a side view of the present invention.

FIG. 4 shows a side view of the present invention that is applied on a spine.

### DETAILED DESCRIPTION OF THE PREFERRED EMBODIMENTS

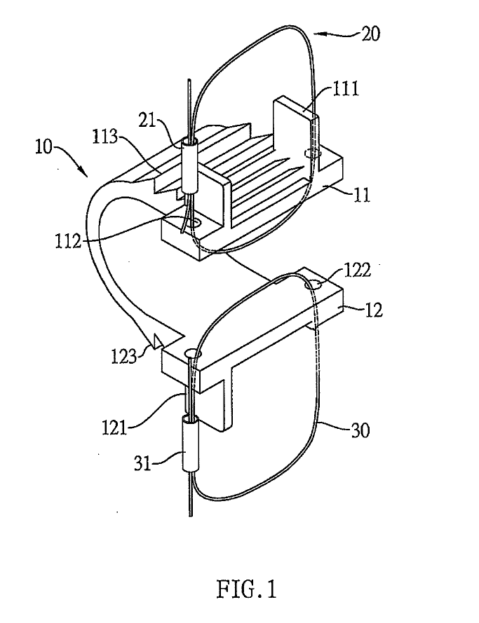

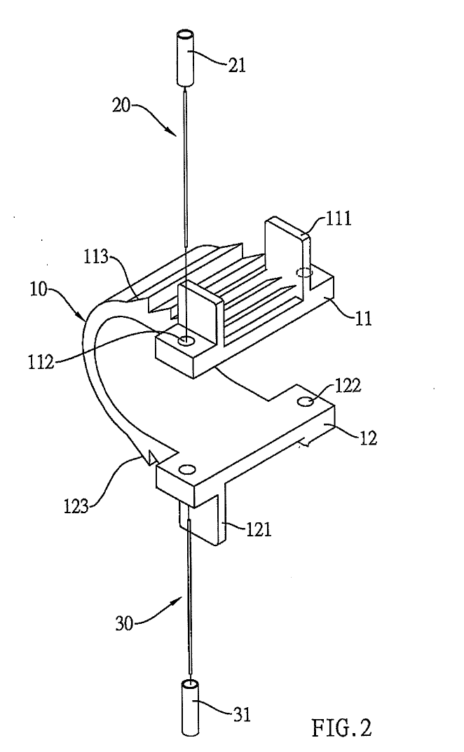

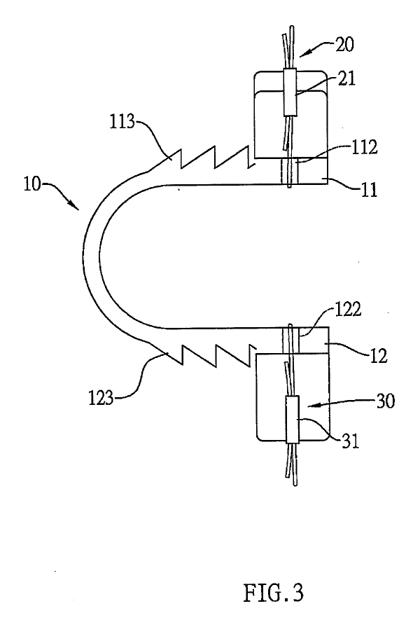

Referring to FIGS. 1 to 4, an adjustable interspinous process distraction device, according to the present invention, comprises a curved plate 10, an upper fixing wire 20 and a lower fixing wire 30.

The curved plate 10 is formed with an upper plate 11 and an opposite lower plate 12 by bending a board material (such as a thin metallic material). The upper plate 11 is penetrated with two upper wire holes 112, and the lower plate 12 is penetrated with two lower wire holes 122.

End parts and opposite exterior sides of the upper and lower plates 11, 12 are separated and longitudinally protruded with two upper clamp pieces 111 and a lower clamp piece 121, respectively. On the other hand, the opposite exterior sides of the upper and lower plates 11, 12 can be formed with inverted hooks 113, 123, with teeth of the inverted hooks 113, 123 facing toward the end parts of the upper and lower plates 11, 12.

The upper fixing wire 20 can be a metallic wire and its two ends penetrate from bottom to top the two upper wire holes 112 of the upper plate 11 to be tied on a spinous process 91 of a vertebra 90.

The lower fixing wire 30 can be a metallic wire and its two ends penetrate from top to bottom the two lower wire holes 122 of the lower plate 12 to be tied on the spinous process 91 of the vertebra 90.

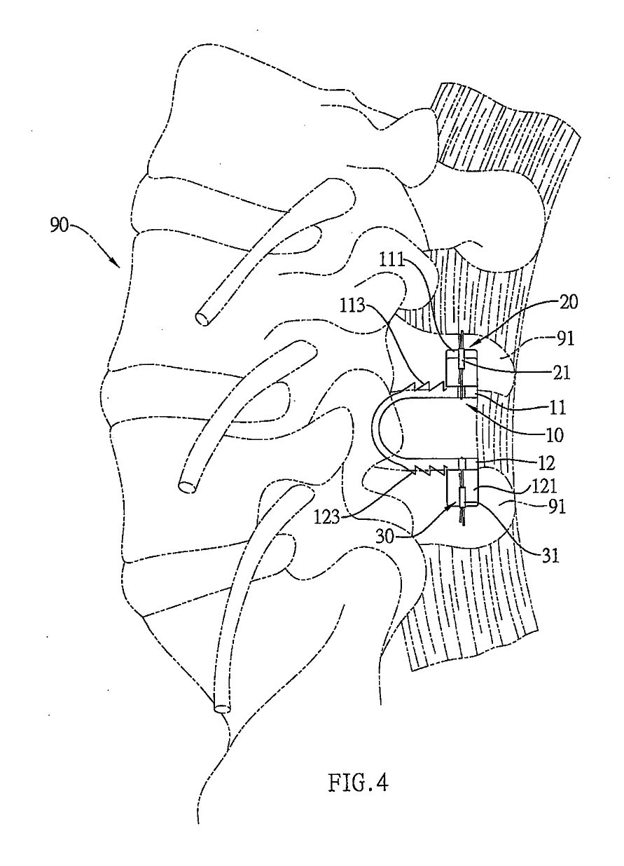

Referring to FIG. 4, each vertebra 90 is provided with a spinous process 91 relative to a rear side of a human body and a gap formed between the two adjacent spinous processes 91 is roughly a trapezoid with a wider front part and a narrower rear part. Therefore, when the present invention is implanted into the gap between the two adjacent spinous processes 91, elasticity of the curved plate 10 can be first utilized to enable the upper and lower plates 11, 12 to be closer to each other, relatively. After spacing between the upper and lower plates 11, 12 has been decreased, the present invention can be then successfully placed between the spinous processes 91 of the two adjacent vertebrae 90. The restoring elasticity provided by the curved plate 10 can allow the upper plate 11 to be abutted at a lower end of one spinous process 91 and one spinous process 91 to be latched between the two upper clamp pieces 111; whereas, the lower plate 12 is abutted at an upper end of the other spinous process 91 and the spinous process 91 is latched in the lower clamp piece 121. Next, the upper fixing wire 20 and the lower fixing wire 30 are used to wrap around the corresponding spinous processes 91 and then tie and position the upper and lower clamp pieces 111, 121 with the spinous processes 91. Besides, the inverted hooks 113, 123 can be gnawed at bone surfaces of the spinous processes 91 to further prevent the curved plate 10 from looseness.

In a preferred embodiment of the present invention, two ends of the upper fixing wire 20 and the lower fixing wire 30 are sheathed respectively with a wire sleeve 21, 31 to fix the two ends, thereby preventing the upper fixing wire 20 or the lower fixing wire 30 from getting released.

The adjustable interspinous process distraction device provided by the present invention is at least provided with following advantages and functional improvements:
1. Compared to the prior art where many vertebrae are tied and positioned together, the present invention only positions two adjacent spinous processes at a time; therefore, a movement between the vertebrae, excessive stiffness of the spine or a pain which can be easily formed at the spine by a slight movement can be avoided, thereby providing better comfortableness.
2. Compared to the existing interspinous process distraction device, the present invention eliminates the structures of screw hole and stud; therefore, the high cost due to that the structures are too complicated can be avoided. In addition, tooth collapse to the screw hole under many times of adjustment can be prevented.
3. The present invention can provide the stable support to two adjacent vertebrae, thereby achieving an object of intensifying the skeleton structure.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claim is:

1. An interspinous process distraction device, comprising:
a curved plate which is formed with an upper plate and an opposite lower plate by bending a board material, with the upper plate being penetrated with two upper wire holes and the lower plate being penetrated with two lower wire holes;
an upper fixing wire, two ends of which penetrate from bottom to top the two upper wire holes of the upper plate to be tied on a spinous process; and
a lower fixing wire, two ends of which penetrate from top to bottom the two lower wire holes of the lower plate to be tied on the spinous process.

2. The interspinous process distraction device, according to claim 1, wherein end parts and opposite exterior sides of the upper and lower plates are separated and protruded respectively with two upper clamp pieces and a lower clamp piece to be clamped on the spinous processes.

3. The interspinous process distraction device, according to claim 1, wherein the exterior sides of the upper and lower plates are formed with inverted hooks.

4. The interspinous process distraction device, according to claim 1, wherein two ends of the upper fixing wire are sheathed with a wire sleeve and two ends of the lower fixing wire are sheathed with a wire sleeve.

5. The interspinous process distraction device, according to claim 2, wherein two ends of the upper fixing wire are sheathed with a wire sleeve and two ends of the lower fixing wire are sheathed with a wire sleeve.

6. The interspinous process distraction device, according to claim 3, wherein two ends of the upper fixing wire are sheathed with a wire sleeve and two ends of the lower fixing wire are sheathed with a wire sleeve.
